(19) United States
(12) Patent Application Publication
(10) Pub. No.: US 2016/0114130 A1
(43) Pub. Date: Apr. 28, 2016

(54) MEDICAL DEVICE INCLUDING A MARKER ELEMENT

(71) Applicant: BOSTON SCIENTIFIC SCIMED, INC., MAPLE GROVE, MN (US)

(72) Inventor: TERRY V. BROWN, FRIDLEY, MN (US)

(73) Assignee: BOSTON SCIENTIFIC SCIMED, INC., MAPLE GROVE, MN (US)

(21) Appl. No.: 14/921,634

(22) Filed: Oct. 23, 2015

Related U.S. Application Data
(60) Provisional application No. 62/068,360, filed on Oct. 24, 2014.

(51) Int. Cl. A61M 25/01; A61M 25/00

(52) U.S. Cl. CPC A61M 25/0108; A61M 25/0012; A61M 25/001

(57) ABSTRACT

During manufacture a marker element is formed over a selected region of a catheter body. A length of heat shrink tubing is disposed over the selected region of the catheter body including the marker element. The catheter assembly is heated causing the heath shrink tubing to constrict in at least a longitudinal direction, forming a mechanical interlock between the marker element and the catheter body at least at one end of the marker element. The heat shrink tubing is later removed. A similar process may be used when bonding a distal tip to a distal end of the catheter body. This process may be used to provide a low profile catheter body even after a marker element has been added.

### CROSS-REFERENCE TO RELATED APPLICATIONS

This application claims priority under 35 U.S.C. §119 to U.S. Provisional Application Ser. No. 62/068,360, filed Oct. 24, 2014, the entirety of which is incorporated herein by reference.

### TECHNICAL FIELD

The present disclosure pertains to medical devices, and methods for manufacturing medical devices. More particularly, the present disclosure pertains to elongated intracorporeal medical devices including a tubular body incorporating other elements, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The present disclosure pertains to elongated intracorporeal medical devices including a tubular body incorporating other elements, and methods for manufacturing and using such devices.

In one example, a catheter includes an elongate body extending from a proximal end to a distal end, the elongate body comprising a polymeric material. A marker element is disposed over a portion of the elongate body, and a first strip of material is disposed adjacent to and in contact with a first end of the marker element. The first strip of material is formed from the polymeric material of the elongate body and defines a mechanical interlock between the marker element and the elongate body.

Alternatively, or additionally, in another example, the elongate body is a multi-layered elongate tubular body having an outer layer comprising the polymeric material. Alternatively, or additionally, in another example, the elongate body further includes an inner layer and a metal braid disposed between the outer layer and the inner layer.

Alternatively, or additionally, the elongate body includes at least a proximal portion, a distal portion and a middle portion disposed between the proximal and distal portion, wherein the distal portion includes the polymeric material, and wherein the marker element is disposed over the distal portion of the elongate body.

Alternatively, or additionally, in another example, a flexibility of the elongate body increases from a proximal end to a distal end of the tubular body.

Alternatively, or additionally, in another example, a second strip of material is disposed adjacent to and in contact with a second end of the marker element, wherein the second strip of material is also formed from the polymeric material of the elongate body and contacts the second end of the marker element to further define a mechanical interlock between the marker element and the elongate body.

Alternatively, or additionally, in another example, a distal tip having a proximal end is disposed adjacent a second end of the marker element, wherein the proximal end of the distal tip abuts the second end of the marker band defining a bumper formed from a portion of the distal tip.

Alternatively, or additionally, in another example, the proximal end of the distal tip abuts the second end of the marker band and has an outer diameter approximately equal to or less than the marker band.

Alternatively, or additionally, in another example, the marker element is radiopaque.

Alternatively, or additionally, in another example, the marker element is a radiopaque metal marker band that circumscribes the elongate body.

Alternatively, or additionally, in another example, the marker element is a radiopaque cuff that partially circumscribes the elongate body.

In another example, a method of securing a marker element to an elongate body includes: disposing a marker element on a selected region of the elongate body, the elongate body including a polymeric material; disposing a heat shrink tubing over the selected region of the elongate body including the marker element, wherein the heat shrink tubing extends past at least a first end of the marker element; and heating the elongate body including the heat shrink tubing disposed over marker element to soften the polymeric material and to constrict the heat shrink tubing in at least a longitudinal direction towards a center line of the marker element. The softening of the polymeric material and constriction of the heat shrink tubing forms at least a first strip of material disposed adjacent to and in contact with a first end of the marker element, wherein the first strip of material is formed from the polymeric material of the elongate body defining a mechanical interlock between the marker element and the elongate body.

Alternatively, or additionally, in another example, the method further including positioning a distal tip element comprising a polymeric material adjacent a second end of the marker element, wherein the heat shrink tubing overlaps a proximal end of the distal tip and wherein heating the tubular body including the heat shrink tubing further causes the polymeric material of the distal tip element to soften and abut the second end of the marker element forming a mechanical interlock between the second end of the marker element and the proximal end of the distal tip element.

Alternatively, or additionally, in another example, the heat shrink tubing further extends past a second end of the marker body, wherein the softening of the polymeric material and the constriction of the heat shrink tubing in response to heating forms at a second strip of material disposed adjacent to and in contact with a second end of the marker element, wherein the second strip of material is formed from the polymeric material of the tubular body and defines a mechanical interlock between the marker element and the elongate body.

Alternatively, or additionally, in another example, the step of forming the marker element comprises swaging the marker element onto the selected region of the elongate body.

In yet another example, a medical device includes an elongate body extending from a proximal end to a distal end and comprising a polymeric material; a marker element disposed on a selected portion of the elongate body; and a first strip of material disposed adjacent to and in contact with a first end of the marker element, wherein the first strip of material is formed from the polymeric material of the elongate body and contacts the first end of the marker element to engage the marker element.

Alternatively, or additionally, in another example, the medical device further includes a second strip of material disposed adjacent to and in contact with a second end of the marker element, wherein the second strip of material is also formed from the polymeric material of the elongate body and contacts the second end of the marker element so as to further engage the marker element.

Alternatively, or additionally, in another example, the medical device further includes a distal tip having a proximal end disposed adjacent a second end of the marker element, wherein the proximal end of the distal tip abuts the second end of the marker element defining a bumper formed from a portion of the distal tip.

Alternatively, or additionally, in another example, the distal tip has as an outer diameter approximately equal to or less than the marker element.

Alternatively, or additionally, in another example, the marker element is radiopaque.

Alternatively, or additionally, in another example, the marker element is a radiopaque metal marker band that circumscribes a distal portion of the elongate body.

Alternatively, or additionally, in another example, the marker element is a radiopaque cuff that partially circumscribes the distal portion of the elongate tubular body.

Alternatively, or additionally, in another example, the marker element is a radiopaque coil disposed over a distal portion of the elongate body.

In yet another example, a catheter includes: a multi-layered elongate tubular body extending from a proximal end to a distal end, the multi-layered, elongate tubular body comprising an outer layer formed from a polymeric material; a marker element disposed over a portion of the elongate tubular body including the outer layer; and a first strip of material disposed adjacent to and in contact with a first end of the marker element, wherein the first strip of material is formed from the polymeric material of the outer layer of the multi-layered tubular body defining a mechanical interlock between the marker element and the portion of the elongate tubular body over which it is disposed.

Alternatively, or additionally, in another example, a flexibility of the elongate tubular body increases from a proximal end to a distal end of the tubular body.

Alternatively, or additionally, in another example, the multi-layered elongate tubular body further including an inner layer and a metal braid disposed between the outer layer and the inner layer.

Alternatively, or additionally, in another example, the catheter further including a second strip of material disposed adjacent to and in contact with a second end of the marker element, wherein the second strip of material is also formed from the polymeric material of the outer layer and contacts the second end of the marker element to further define a mechanical interlock between the marker element to the portion of the elongate tubular body over which it is disposed.

Alternatively, or additionally, in another example, the catheter further including a distal tip having a proximal end disposed adjacent a second end of the marker element, wherein the proximal end of the distal tip abuts the second end of the marker band defining a bumper formed from a portion of the distal tip so as to mechanically interlock the proximal end of the distal tip with the second end of the marker element.

Alternatively, or additionally, in another example, the proximal end of the distal tip abuts the second end of the marker element and has an outer diameter approximately equal to or less than the marker element.

Alternatively, or additionally, in another example, the marker element is radiopaque.

Alternatively, or additionally, in another example, the marker element is a radiopaque metal marker band that circumscribes the distal portion of the elongate tubular body

In still yet another example, a method of securing a marker element to an elongate body includes: disposing a marker element on a selected region of the elongate body, the elongate body including a polymeric material; disposing a heat shrink tubing over the selected region of the tubular body including the marker element, wherein the heat shrink tubing extends past at least a first end of the marker body; and heating the elongate body including the heat shrink tubing disposed over marker element to soften the polymeric material and to constrict the heat shrink tubing in at least a longitudinal direction towards a center line of the marker element, wherein softening of the polymeric material and constriction of the heat shrink tubing forms at least a first strip of material disposed adjacent to and in contact with a first end of the marker element, wherein the first strip of material is formed from the polymeric material of the elongate body defining a mechanical interlock between the marker element and the selected region of the elongate body.

Alternatively, or additionally, in another example, the method further includes positioning a distal tip element comprising a polymeric material adjacent a second end of the marker element, wherein the heat shrink tubing overlaps a proximal end of the distal tip and wherein heating the tubular body including the heat shrink tubing further causes the polymeric material of the distal tip element to soften and abut the second end of the marker element forming a mechanical interlock between the second end of the marker element and the proximal end of the distal tip element.

Alternatively, or additionally, in another example, the heat shrink tubing further extends past a second end of the marker element, wherein the softening of the polymeric material and the constriction of the heat shrink tubing in response to heating forms a second strip of material disposed adjacent to and in contact with a second end of the marker element, wherein the second strip of material is formed from the polymeric material of the elongate body and defines a mechanical interlock between the marker element and the selected region of the elongate body.

Alternatively, or additionally, in another example, the step of forming the marker element includes swaging the marker element onto the selected region of the elongate body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view of an exemplary medical device;

FIG. 2 is a cross-sectional view of the medical device shown in FIG. 1 taken through line 2-2;

FIG. 3 is a schematic view of a portion of a catheter body including a marker element;

FIG. 4 is a longitudinal cross-sectional view of the portion of the catheter body shown in FIG. 3 taken through line 4-4;

FIG. 5 is a close-up, schematic view of the a portion of the catheter body shown in FIG. 3;

FIGS. 6A-6E provide an illustrative representation of the steps in a method of forming an interlocking structure between a marker element and the catheter body;

FIG. 7 is a flow chart of the method corresponding to the illustrative steps shown in FIGS. 6A-6E;

FIG. 8 is a schematic view of another exemplary medical device;

FIG. 9 is a close-up schematic view of a distal end region of the catheter shown in FIG. 8;

FIG. 10 is a longitudinal cross-sectional view of the distal end region of the catheter shown in FIG. 9;

FIGS. 11A-11F provide an illustrative representation of the steps in a method of forming an interlocking structure between a marker element, the catheter body, and a distal tip; and

FIG. 12 is a flow chart of the method corresponding to the illustrative steps shown in FIGS. 11A-11E.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical devices, such as, but not limited to, catheters, may utilize one or more marker elements or marker bands to aid in the visualization of the device (via any number of known visualization techniques) while the medical device is in use within a body. In some instances, the marker bands are held in place using a retention sleeve. For example, the marker band may be swaged onto a tubular element and a retention sleeve placed over the marker band. A length of heat shrink tubing may be placed over the retention sleeve and heated to a temperature slight above a melting point temperature of the material forming the retention sleeve. This may allow the retention sleeve and/or an outer surface of the tubular element to soften. The heat shrink tubing, retention sleeve and tubular element may be allowed to cool until the polymeric material of the retention sleeve and/or tubular element has hardened. The heat shrink tubing may then be removed. The retention sleeve may secure the marker band to the tubular element. However, the retention sleeve has a thickness which may contribute to the overall diameter of the device. For example, a retention sleeve having a wall thickness of 0.0015 inches (0.0381 millimeters) may increase the overall diameter of the device adjacent to the maker band by 0.003 inches (0.0762 millimeters). It may be desirable to secure a marker band to a medical device without the use of a retention sleeve.

It is further contemplated that the ability to secure a marker band to a medical device without the use of a retention sleeve may have manufacturing advantages. For example, the manufacturing cycle time may be reduced by removing the step of aligning the retention sleeve over the marker band. For example, the assembler (operator) would not have to align the retention sleeve over the marker band and then put the heat shrink tubing over the sleeve without moving the retention sleeve which can take one or more attempts to achieve. It is further contemplated that the ability to secure a marker band to a medical device without the use of a retention sleeve may also reduce defective assemblies that are not useable or scrap materials. For example, in some instances, the retention sleeve may include bubbles or be found to be misaligned after the heat shrink process. The removal of the retention sleeve may also reduce the material costs associated with the device.

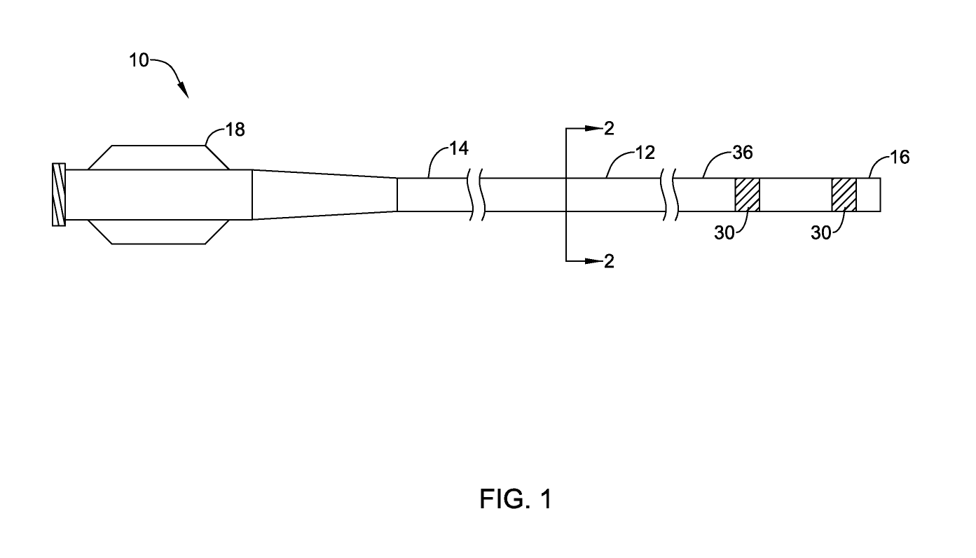

FIG. 1 is a side schematic view of an exemplary medical device 10. Although medical device 10 is described as a guide catheter, device 10 could be any other type of catheter including diagnostic or therapeutic catheters such as angioplasty balloon catheters, atherectomy catheters, stent delivery catheters, and the like, or any other suitable device.

Furthermore, medical device 10 can generally include any device designed to pass through an opening or body lumen. For example, medical device 10 may comprise an endoscopic device, laproscopic device, embolic protection device, guidewire and the like, or any other suitable device.

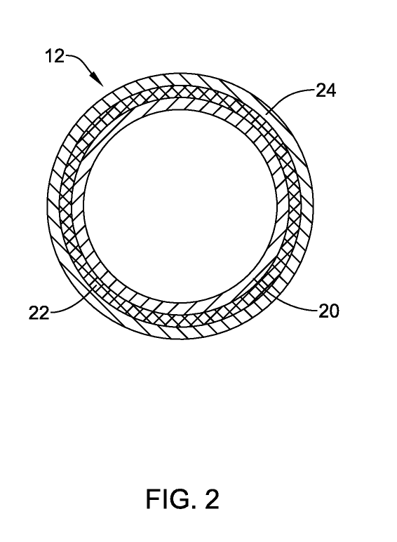

Catheter 10 may include an elongated catheter body 12 extending from a proximal end region 14 to a distal end region 16. A hub 18 may be coupled to proximal end region 14. In at least some embodiments, elongated catheter body 12 may include a plurality of layers. For example, FIG. 2 illustrates that elongated catheter body 12 may include an inner liner or layer 20, a reinforcing layer 22, and an outer layer 24. Liner 20 may include lubricious material such as polytetrafluoroethylene (PTFE), etched PTFE, fluorinated ethylene propylene (FEP), or the like. Outer layer 24 may include one or more polymers such as polyether block amide, polyurethane, combinations or blends thereof, or the like. All of the layers 20, 22, 24 may extend along the full length of elongated catheter body 12. Alternatively, one or more of layers 20, 22, 24 may extend along only a portion of the length of elongated catheter body 12.

Reinforcing layer 22 may include a braid, coil, mesh, or other suitable reinforcement. In at least some embodiments, reinforcing layer 22 may include a polymeric braid. For example, reinforcing layer 22 may include an ultra-high molecular weight polyethylene braid. Other materials and/or reinforcements are contemplated including those disclosed herein. The presence of reinforcing layer 22 may provide elongated catheter body 12 with enhanced cut resistance, tear resistance, kink resistance, etc.

Alternatively and/or additionally, the elongated catheter body 12 may be manufactured such that it increases in flexibility along a length of the elongated body 12 from the proximal end 14 to the distal end 16. For example, in such an embodiment, a distal portion of the catheter body 12 may have a greater flexibility than a middle and/or proximal portion of the catheter body 12. Similarly, the middle portion may have less flexibility than the distal portion, but greater flexibility than the proximal portion. A variable flexibility profile may be achieved by manipulating the material properties and/or the mechanical/structural properties of the catheter body 12.

A number of different methods may be used to manufacture elongated catheter body 12. For example, liner 20 may be disposed on a mandrel. The mandrel may vary in size, depending on the intervention. For example, the mandrel may be a silver coated copper core or other suitable mandrel with an outer diameter in the range of about 0.01 to 0.05 inches (about 0.25 millimeters to 1.27 millimeters), or about 0.02 to 0.04 inches (about 0.51 millimeters to 1.02 millimeters), or about 0.022 to 0.027 inches (about 0.559 millimeters to 0.686 millimeters) or so. In some embodiments, reinforcing layer 22 may be disposed along the outer surface of liner 20 and outer layer 24 may be disposed along the outer surface of reinforcing layer 22. It is contemplated that while reinforcing layer 22 is described as an intermediate layer, the reinforcing layer 22 may be an innermost layer of the catheter body or an outermost layer of the catheter body 12, as desired. For example, in some embodiments, outer layer 24 may be disposed along the outer surface of liner 20 and reinforcing layer 22 may be disposed along the outer surface of outer layer 24. The process for disposing layers 20, 22, 24 onto the mandrel may include an extrusion process. When using an extrusion process, the medical device assembly may be subjected to extrusion temperatures in the range of about 100 to 200° C., or about 120 to 190° C., or about 140 to 170° C. Under such conditions, reinforcing layer 22 may become embedded and/or at least partially embedded within outer layer 24. For example, at least a portion of outer layer 24 may be disposed radially outward of the outer surface of reinforcing layer 22. In some instances, reinforcing layer 22 may become disposed at or near the inner surface of outer layer 24 so that reinforcing layer 22 is essentially positioned between liner 20 and outer layer 24. In some of these and in other embodiments, portions of outer layer 24 may be interlocked with or otherwise disposed within the interstices of reinforcing layer 22. This may form or define a “composite layer” that includes both the material of reinforcing layer 22 and the material of outer layer 24. In at least some embodiments, the melting temperature of reinforcing layer 22 may be less than the melting temperature of liner 20, outer layer 24, or both. This is just one example.

Referring back to FIG. 1, catheter 10 may also include one or more marker elements 30 that is/are coupled to the distal end region 16 of the elongated catheter body 12. Marker elements 30 may be disposed anywhere along the length of the body 12. Multiple marker elements 30 may be distributed along the length of the body 12. In at least some embodiments, at least one marker element 30 may be located on a distal portion 36 of the body 12. The marker element 30 may be incorporated into the catheter 10 as a cuff, coil, or band that at least partially circumscribes the body 12. In cases where the marker element 30 is a band or coil, the marker element 30 may fully circumscribe the body 12.

Generally, the purpose for including marker element 30 as a part of catheter 10 or any other medical device is to aid in the visualization of catheter 10 (via any number of known visualization techniques) while the medical device is in use within a body. Typically the visualization techniques used rely on marker element 30 being made from or otherwise including a radiopaque material. Radiopaque materials are understood to be materials capable of producing a relatively bright image on a fluoroscopy screen or another imaging technique during a medical procedure. This relatively bright image aids the user of catheter 10 in determining its location. Some examples of radiopaque materials can include, but are not limited to, gold, platinum, platinum-iridium, palladium, tantalum, tungsten alloy, plastic material loaded with a radiopaque filler, and the like. In at least some of the embodiments, the marker element 30 is fabricated from a radiopaque metal or metal alloy.

Marker element 30 may be incorporated into catheter 10 by disposing marker element 30 over catheter body 12 during the manufacturing process. Securing the position of marker element 30 may be important for a number of reasons. For example, if marker element 30 is properly secured, the clinician can rely on the known position of marker element 30 in order to accurately assess the position of the remainder of catheter 10. This may include the clinician being able to know the precise location of the distal end of catheter 10 by virtue of visualizing marker element 30 and knowing how far the marker element 30 is from the distal end of catheter 10. This may be critically important when catheter 10 is being used in a particular sensitive location, such as the central nervous system, because errantly positioning the catheter 10 could damage sensitive areas. It can be appreciated that given the small scale of catheters and blood vessel, even a very small shift in the position of marker element 30 may have an impact on a medical intervention.

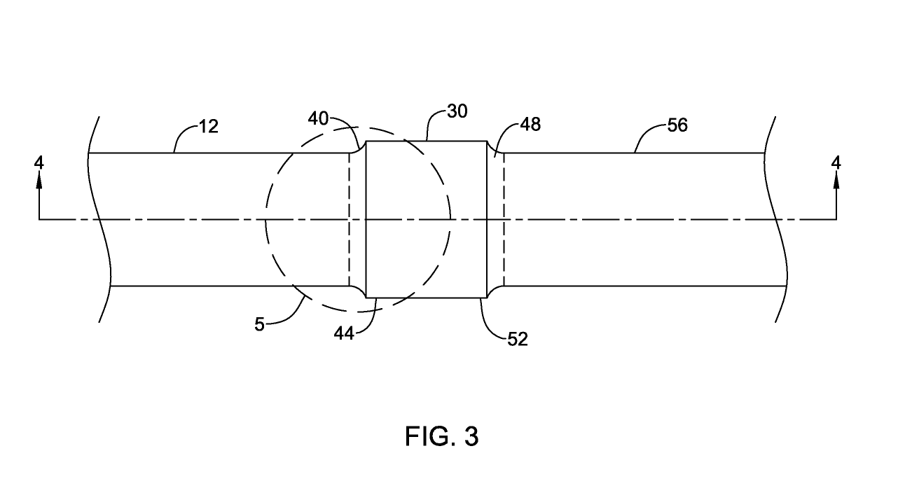

During fabrication, the marker element 30 may be secured relative to catheter body 12 by forming an interlocking structure between the marker element 30 and the polymeric material forming a portion of the catheter body 12 such as, for example, the polymeric material forming the outer layer 24 of a multi-layered catheter body 12. FIGS. 3-5 show different views of marker element 30 secured to the catheter body 12 by at least one interlocking structure. In at least some embodiments, the interlocking structure may be formed as a thin strip of material or fillet 40 that is disposed adjacent to and in contact with at least one end 44 of the marker element 30. In some cases, the marker element 30 is interlocked with the catheter body 12 by a thin strip of material or fillet 40, 48 disposed adjacent to and in contact with each end 44, 52 of the marker element 30.

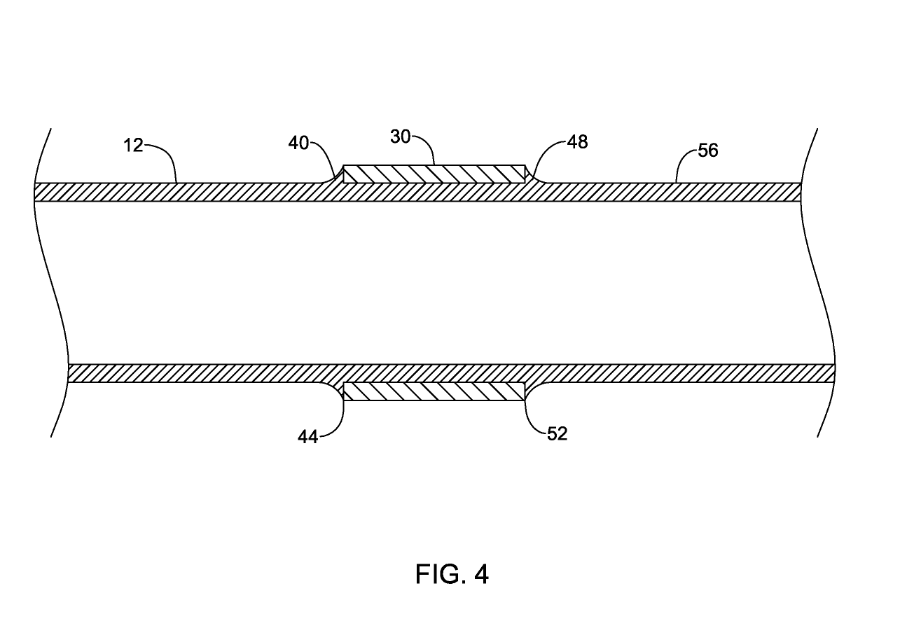

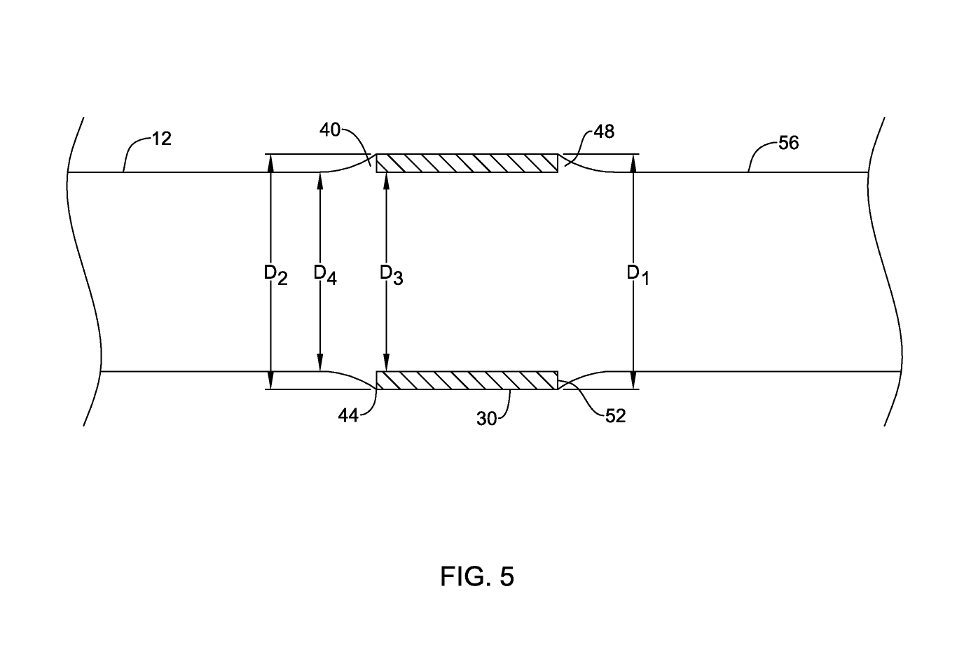

In at least some cases, the outer diameter D₁ (see FIG. 5) of the marker element 30 is substantially equal to or slightly greater than the outer diameter D₂ of the thin strip of polymeric material 40 and/or 48 forming the interlocking structure with the marker element 30 such that the marker element 30 does not substantially protrude above an outer surface 56 of the catheter body 12 so as to maintain the low profile of the catheter body 12 (FIGS. 3 and 4). Additionally, as can be seen in FIG. 5, an outer diameter D₂ of the polymeric strip of material 40 and/or 48 forming the interlocking structure with the marker element 30 is greater than an inner diameter D₃ of the marker element 30. Alternatively, the polymeric strip of material 40 and/or 48 forming the interlocking structure with the marker element 30 has a first outer diameter D₂ and a second outer diameter D₄ wherein the first outer diameter D₂ is greater than the second outer diameter D₄ and the second outer diameter D₄ is substantially equal to the inner diameter D₃ of the marker element 30 so as to provide a secure interlock between the marker element 30 and the catheter body 12.

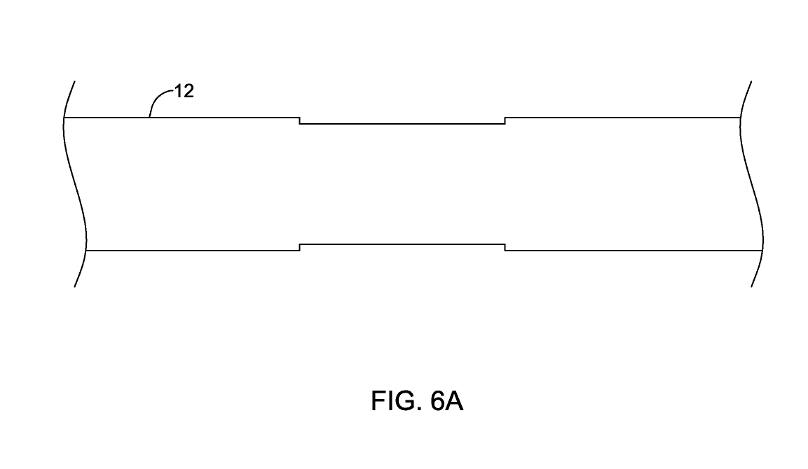

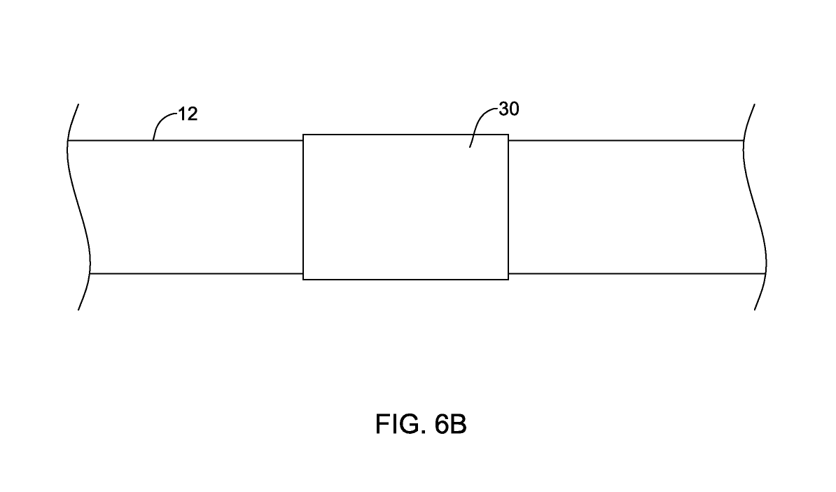

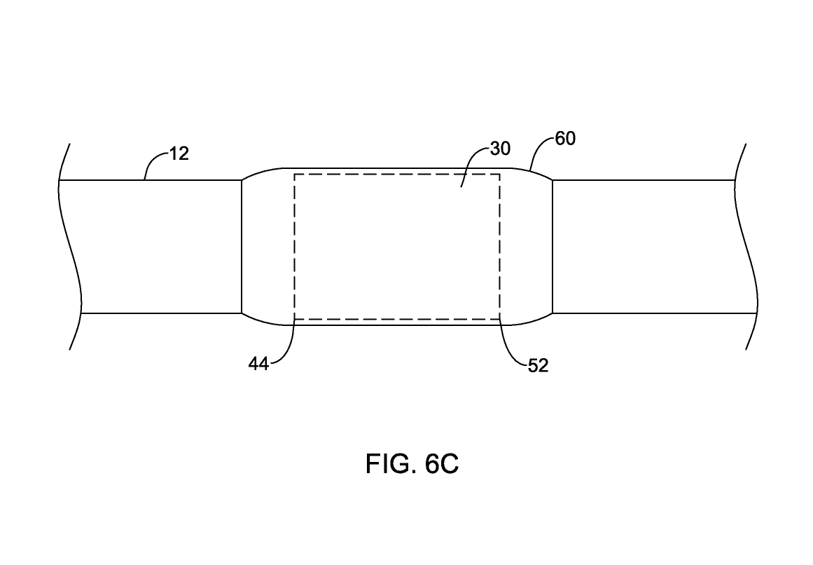

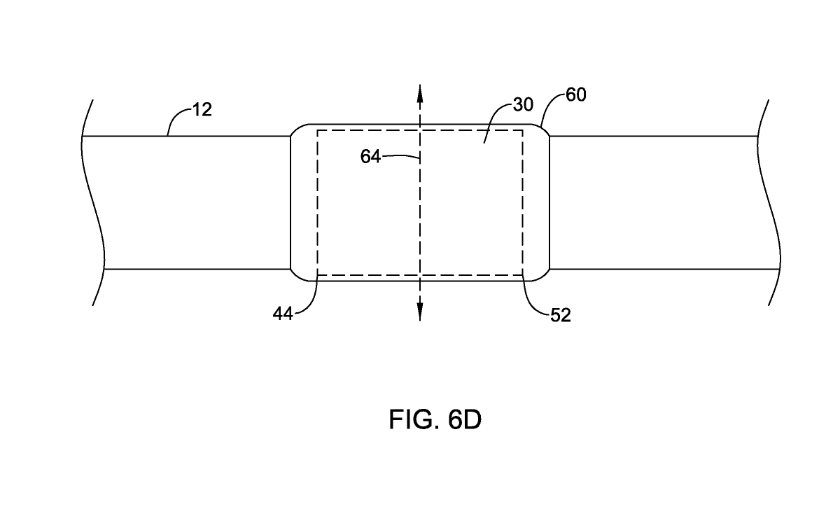

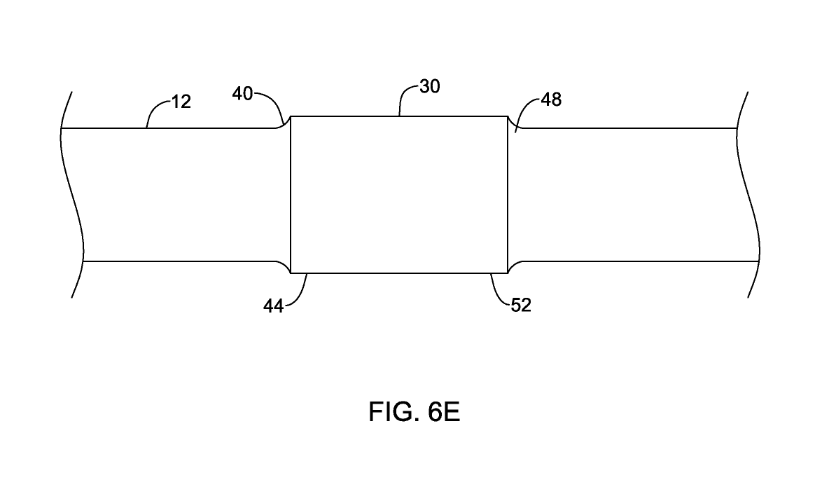

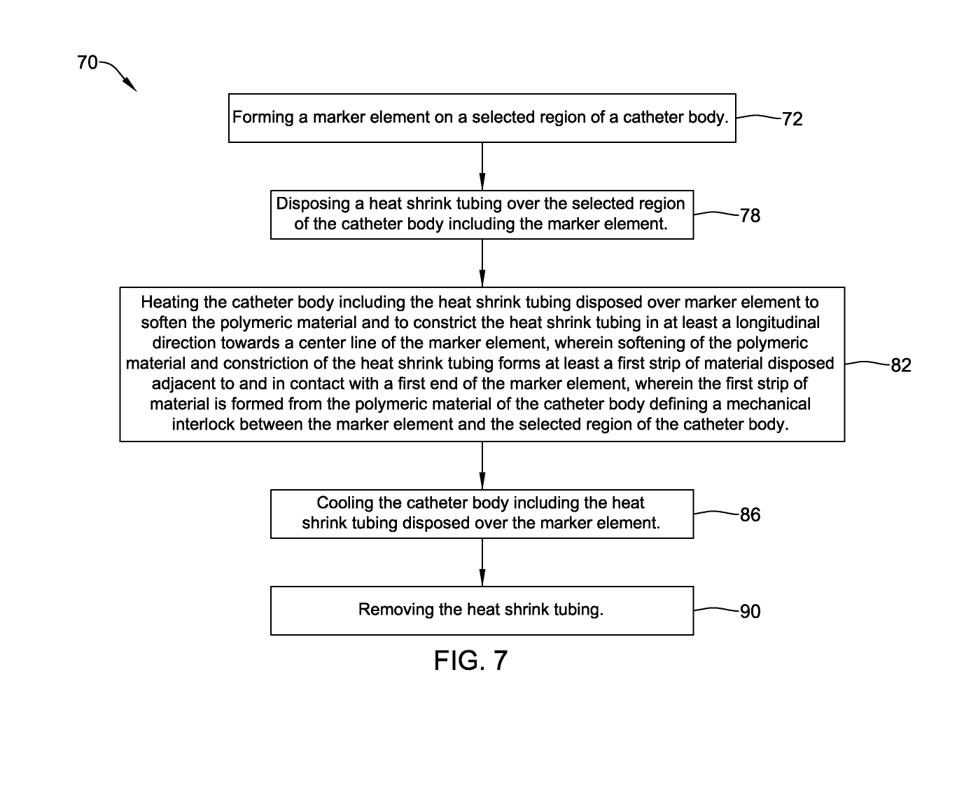

FIGS. 6A-6E and the flow chart of FIG. 7 outline the steps of a method 70 that may be used to create the interlocking structure or arrangement between marker element 30 and the catheter body 12 at a desired location along the length of the catheter. In a first step, represented by FIGS. 6A and 6B, the marker element 30 may be swaged onto the catheter body 12 at a desired location (Block 72 of FIG. 7). A length of commercially available heat shrink tubing 60 is provided over the marker element 30 and catheter body 12 assembly (FIG. 6C and Block 78 of FIG. 7). The length of the heat shrink tubing 60 may be selected such that when placed over the marker element 30 the ends of the heat shrink tubing 60 extend slightly beyond the first and second ends 44, 52 of the marker element 30, as shown in FIG. 6C. The marker element 30 and catheter body 12 assembly are then placed into a clamping device (not shown) and heated to a temperature slightly above a melting point temperature of the polymeric material forming the catheter body 12 such that the polymeric material begins to soften (Block 82). In at least some embodiments, the assembly is heated to a temperature slightly above a melting point temperature of the polymeric material forming an outer layer 24 of the catheter body 12. In addition, during the heating step, the heat shrink tubing 60 begins to shrink, constricting the heat shrink tubing 60 in at least the longitudinal direction towards a center line 64 of the marker element 30, as represented by FIG. 6D. In some cases, the heat shrink tubing 60 may also constrict radially, applying a radial compressive force to the marker element 30 and the underlying, softened catheter body 12. Constriction of the heat shrink tubing 60 in the longitudinal and/or radial direction causes a thin strip of polymeric material to be formed from the catheter body 12. For example, as the polymeric material of the catheter body 12 softens and/or melts the longitudinal and/or radial force of the heat shrink tubing 60 may cause the catheter body 12 material to form a fillet 40, 48 adjacent to the ends 44, 52 of the marker element 30. It also may cause the polymeric material to back fill any remaining empty space between the marker element 30 and the catheter body 12, improving the interface between the marker element 30 and the catheter body 12. The thin strip of material or fillet 40, 48 is formed such that it is adjacent to and in contact with an end 44 or 52 of the marker element 30 creating an interlocking structure between the marker element 30 and the catheter body 12. It will be generally understood that depending upon the desired location of the marker element 30 on the catheter body 12, that a second thin strip of material or fillet 48 can be formed such that it is adjacent to and in contact with the opposite end 44 or 52 of the marker element 30. The marker element 30 and catheter body 12 assembly may then be cooled until the polymeric material is hardened (Block 86 of FIG. 7). This can be done by simply removing the application of heat, allowing the assembly to cool at room temperature, or placing the assembly into a cool water or ice bath. After the assembly has been cooled, the heat shrink tubing may be removed, exposing the marker element 30 and the fillets 40, 48 (FIG. 6E and Block 90 of FIG. 7).

The method 70 to create the interlocking structure or arrangement between marker element 30 and the catheter body 12 at a desired location along the length of the catheter may allow the marker element 30 to be secured to the catheter body 12 without the use of a retention sleeve. This may result in a reduction of the diameter of the device adjacent to the marker element 30 being smaller than that of a device using a retention sleeve. As noted above, securing the marker element 30 without the use of a retention sleeve may have additional manufacturing advantages.

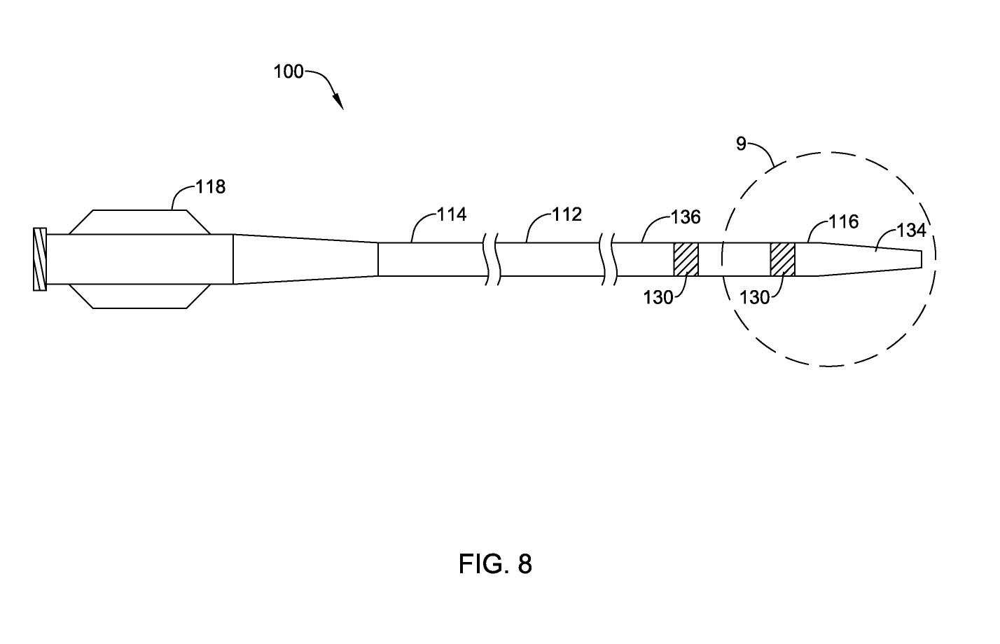

FIG. 8 is a schematic view of another exemplary medical device 100. Although medical device 100 is described as a guide catheter, device 100 could be any other type of catheter including diagnostic or therapeutic catheters such as angioplasty balloon catheters, atherectomy catheters, stent delivery catheters, and the like, or any other suitable device. Furthermore, medical device 100 can generally include any device designed to pass through an opening or body lumen. For example, medical device 100 may comprise an endoscopic device, laproscopic device, embolic protection device, guidewire and the like, or any other suitable device.

Similar to catheter 10, described herein, catheter 100 may include an elongated catheter body 112 extending from a proximal end region 114 to a distal end region 116. A hub 118 may be coupled to proximal end region 114. In at least some embodiments, similar to catheter body 12, elongated catheter body 112 may also include a plurality of layers, as described herein in reference to FIG. 2. Catheter 100 may also include one or more marker elements 130, as described herein with reference to FIGS. 1-7, and/or a distal tip 134 that is coupled to the distal end region 116 of the elongated catheter body 112. Marker elements 130 may be disposed anywhere along the length of the body 112. In at least some embodiments, at least one marker element 130 may be located on a distal portion 136 of the body 112. The marker elements 130 may be incorporated into the catheter 100 as a cuff, coil, or band that at least partially circumscribes the body 112. In cases where the marker element 130 is a band or coil, the marker element 130 may fully circumscribe the body 112.

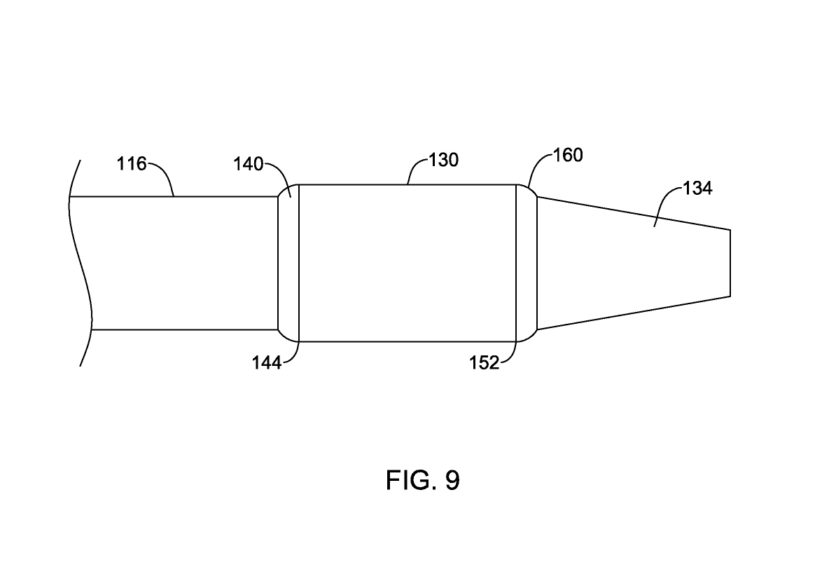

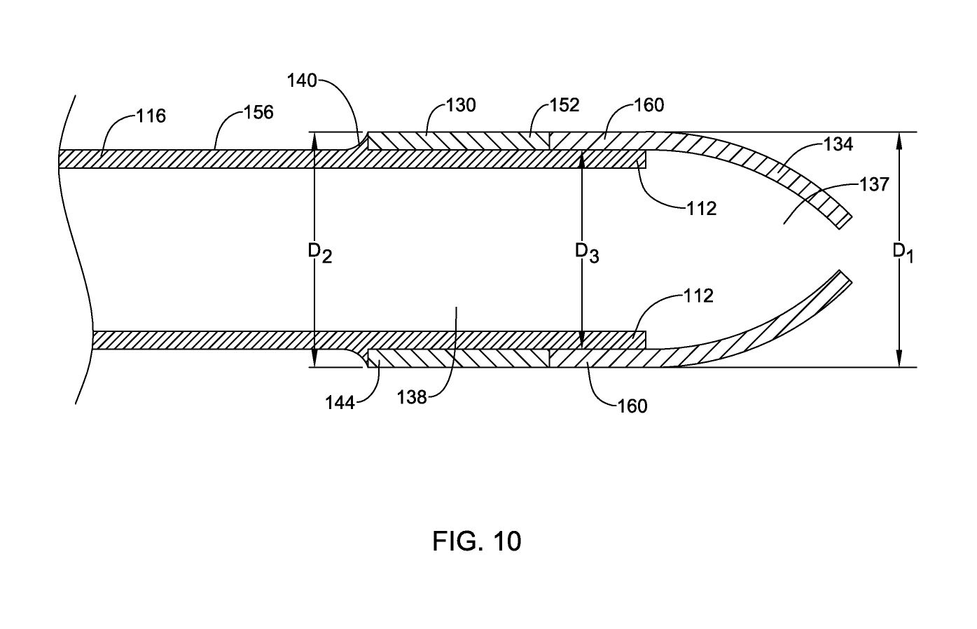

FIGS. 9 and 10 are close-up schematic views of the distal end region 116 of the catheter body 112 including a marker element 130 and a distal tip 134. The distal tip 134 may be a soft distal tip 134 and may be fabricated from a polymeric material having a lower durometer than the polymeric material used to form at least one layer of the catheter body 112. In at least some cases, the distal tip 134 may include a lumen through which other devices and/or fluid may pass, depending upon the desired application. As shown in FIG. 10, the distal tip lumen 137 is in communication with a main body lumen 138 of the catheter body 112. The distal tip 134 may be bonded to or otherwise coupled to a distal end region 116 of the catheter body 112 using a similar process described above with reference to FIGS. 6A-7. As described herein, a first thin strip of material or fillet 140 may be formed at a first end 144 of the marker element 130, securing the marker element 130 to the catheter body 112. In addition, the distal tip 134 is secured to the catheter body 112 such that that it abuts and is in contact with a second end 152 of the marker element 130, further securing the marker element 130 to the catheter body 112. The distal tip 134 may be bonded to the catheter body 112 adjacent the marker element 130 such that it contacts the second end 152 of the marker element and forms a bumper or dam 160, forming an additional interlocking structure between the marker element 130, the catheter body 112, and the distal tip 134.

As described herein, during fabrication, the marker element 130 may be secured relative to catheter body 112 by forming an interlocking structure between the marker element 130 and the polymeric material forming a portion of the catheter body 112 such as, for example, the polymeric material forming an outer layer of a multi-layered catheter body 112, such as outer layer 24 described above. In at least some embodiments, the interlocking structure may be formed as a thin strip of material or fillet 140 that is disposed adjacent to and in contact with at least one end 144 of the marker element 130. The distal tip 134 may be placed over a portion of the catheter body 112 such that it is adjacent to and in contact with a second end 152 of the marker element 130 such that when bonded to the catheter body 112 a second interlocking structure or bumper 160 is formed between the marker element 130, the catheter body 112, and the distal tip 134.

In at least some cases, the outer diameter D₁ of the marker element 130 and/or the bumper 160 of the distal tip 134 is substantially equal to or slightly greater than the outer diameter D₂ of the thin strip of polymeric material 140 forming the interlocking structure at a first end 144 of the marker element 130 such that the marker element 130 and/or the bumper 160 do not substantially protrude above an outer surface 156 of the catheter body 112 so as to maintain the low profile of the catheter body 112. In addition, an inner diameter D₃ of the distal tip 134 may be greater than the outer diameter of the catheter body 112 such that the distal tip 134 is able to be disposed over the catheter body 112 during the manufacturing process.

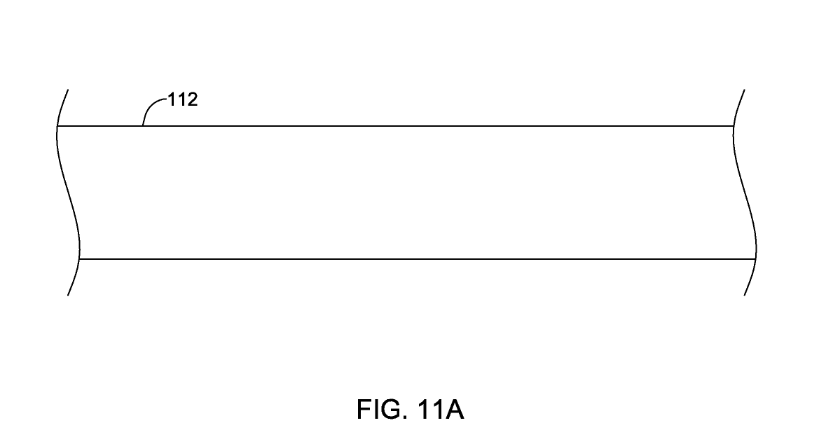

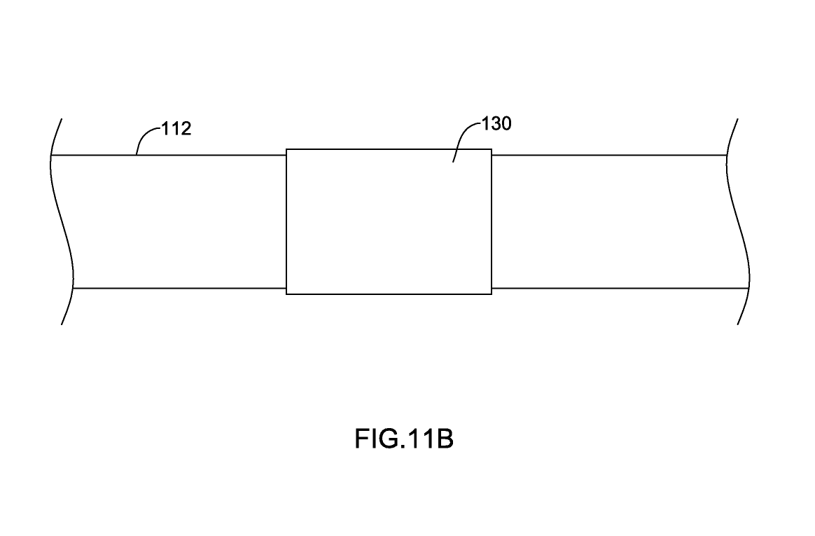

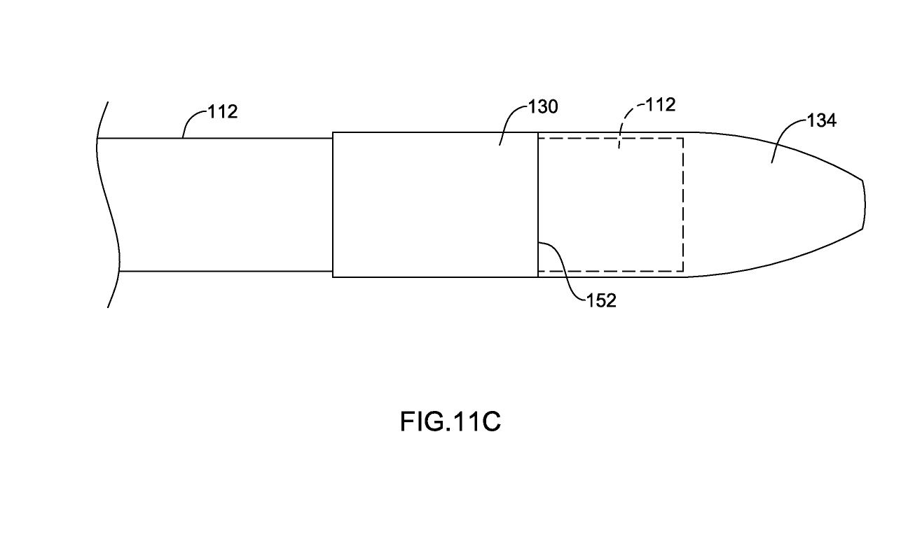

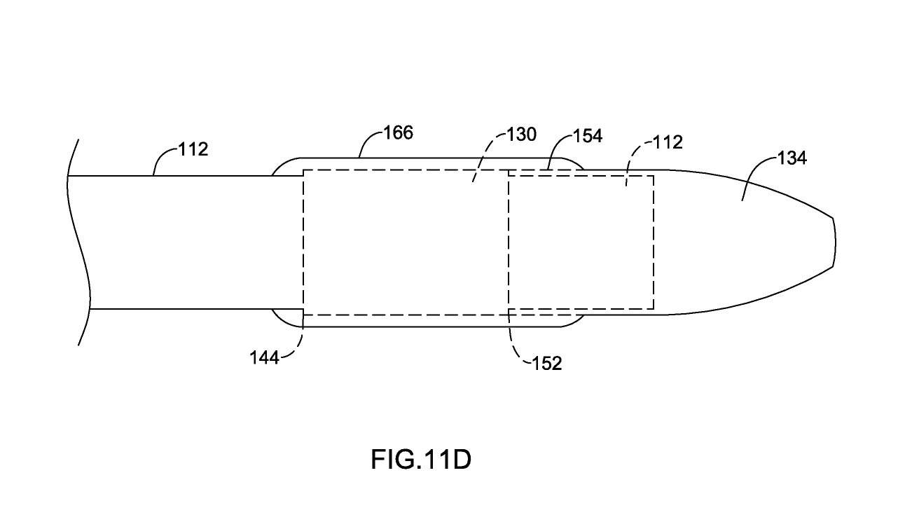

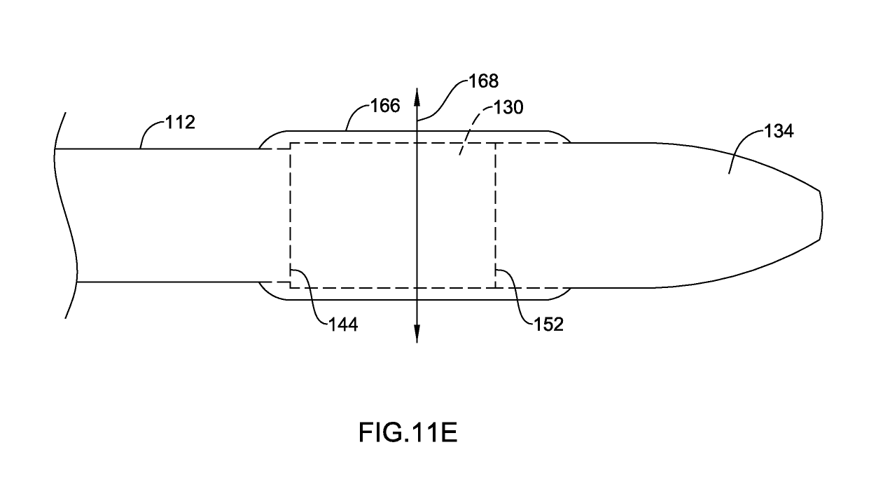

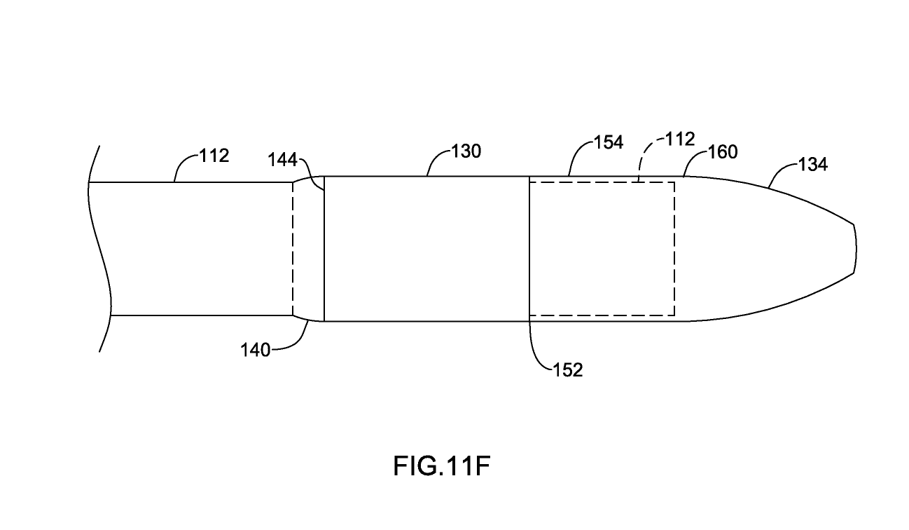

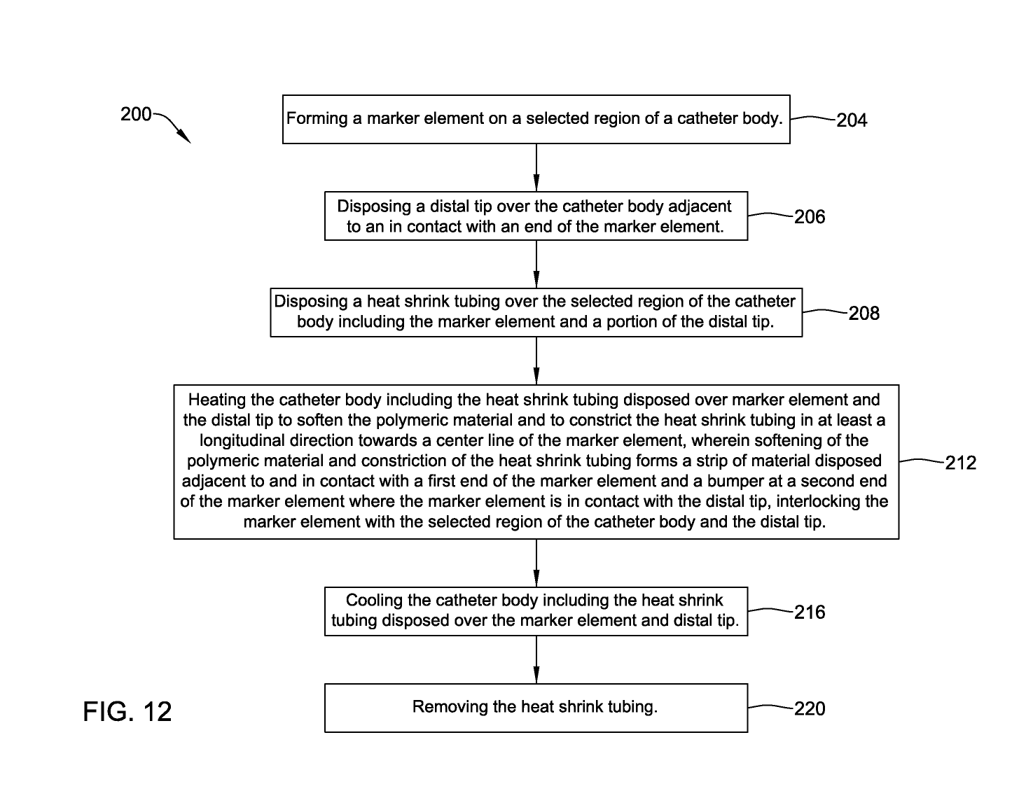

FIGS. 11A-11F and the flow chart of FIG. 12 outline the steps of a method 200 that may be used to create the interlocking structure or arrangement between marker element 130 and the catheter body 112, and also between the marker element 130, the catheter body 112, and the distal tip 134. In a first step, represented by FIGS. 11A and 11B, the marker element 130 may be swaged onto the catheter body 112 at a desired location (Block 204 of FIG. 12). A distal tip 134 is disposed over a distal end of the catheter body 112 that extends beyond a second end 152 of the marker element 130 (FIG. 11C and Block 206 of FIG. 12) such that the distal tip 134 abuts the second end 152 of the marker element 130. Next, a length of commercially available heat shrink tubing 166 is provided over the catheter body 112 including the marker element 130 and at least a proximal portion 154 of the distal tip 134 (FIG. 11D and Block 208 of FIG. 12). The length of the heat shrink tubing 166 may be selected such that when placed over the marker element 130 and the distal tip 134, the ends of the heat shrink tubing 166 extend slightly beyond the first and second ends 144, 152 of the marker element 130 and also over at least a proximal end 154 of the distal tip 134, as shown in FIG. 11D. The catheter body 112 including the heat shrink tubing 166 disposed over the marker element 130 and at least a portion of the distal tip 134 is then placed into a clamping device (not shown) and heated to a temperature slightly above a melting point temperature of the polymeric material forming the catheter body 112 such that the polymeric material begins to soften (Block 212 of FIG. 12). In at least some embodiments, the catheter body assembly is heated to a temperature slightly above a melting point temperature of the polymeric material forming an outer layer of the catheter body 112. In addition, during the heating step, the heat shrink tubing 166 begins to shrink, constricting the heat shrink tubing 166 in at least the longitudinal direction towards a center line 168 of the marker element 130, as represented by FIG. 11E. In some cases, the heat shrink tubing 166 may also constrict radially, applying a radial compressive force to the marker element 130 and the underlying, softened catheter body 112 and the distal tip 134. Constriction of the heat shrink tubing 166 in the longitudinal and/or radial direction causes a thin strip of polymeric material to be formed from the catheter body 112 at the first end 144 of the marker element 130. For example, as the polymeric material of the catheter body 112 softens and/or melts the longitudinal and/or radial force of the heat shrink tubing 166 may cause the catheter body 112 material to form a fillet 140 adjacent to the end 144 of the marker element 130. It also may cause the polymeric material to back fill any remaining empty space between the marker element 30 and the catheter body 112 and also the distal tip 134 and the catheter body, improving the interface between the marker element 130 and the catheter body 112 as well as the distal tip 134 and the catheter body 112. The thin strip of material or fillet 140 is formed such that it is adjacent to and in contact with an end 144 of the marker element 130 creating an interlocking structure between the marker element 130 and the catheter body 112. In addition, a bumper 160 is formed between the distal tip 134 and the second end 152 of the marker element 130. At the bumper 160, the distal tip abuts and is in contact with the second end 152 of the marker element 130. The interface between the marker element 130, the catheter body 112, and the distal tip 134 provides an additional interlocking structure, further securing the marker element 130 and the distal tip 134 to the catheter body 112. The catheter assembly including the catheter body 112, the marker element 130 and the distal tip 134 may then be cooled until the polymeric material is hardened (Block 216 of FIG. 12). This can be done by simply removing the application of heat, allowing the assembly to cool at room temperature, or placing the assembly into a cool water or ice bath. After the assembly has been cooled, the heat shrink tubing 166 may be removed, exposing the marker element 130, the fillet 140, and the distal tip 134 (FIG. 11F and Block 220 of FIG. 12).

The method 200 to create the interlocking structure or arrangement between marker element 130, the catheter body 112, and/or distal tip 134 at a desired location along the length of the catheter may allow the marker element 130 to be secured to the catheter body 112 without the use of a retention sleeve and/or without using distal tip 134 as a retention sleeve. This may result in a reduction of the diameter of the device adjacent to the marker element 130 being smaller than that of a device using a retention sleeve. As noted above, securing the marker element 130 without the use of a retention sleeve may have additional manufacturing advantages.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A medical device comprising:
an elongate body extending from a proximal end to a distal end and comprising a polymeric material;
a marker element disposed on a selected portion of the elongate body; and
a first strip of material disposed adjacent to and in contact with a first end of the marker element, wherein the first strip of material is formed from the polymeric material of the elongate body and contacts the first end of the marker element to engage the marker element.

2. The medical device of claim 1, further comprising a second strip of material disposed adjacent to and in contact with a second end of the marker element, wherein the second strip of material is also formed from the polymeric material of the elongate body and contacts the second end of the marker element so as to further engage the marker element.

3. The medical device of claim 1, further comprising a distal tip having a proximal end disposed adjacent a second end of the marker element, wherein the proximal end of the distal tip abuts the second end of the marker element defining a bumper formed from a portion of the distal tip.

4. The medical device of claim 3, wherein the distal tip has as an outer diameter approximately equal to or less than the marker element.

5. The medical device of claim 1, wherein the marker element is radiopaque.

6. The medical device of claim 1, wherein the marker element is a radiopaque metal marker band that circumscribes a distal portion of the elongate body.

7. The medical device of claim 1, wherein the marker element is a radiopaque cuff that partially circumscribes the distal portion of the elongate tubular body.

8. The medical device of claim 1, wherein the marker element is a radiopaque coil disposed over a distal portion of the elongate body.

9. A catheter comprising:
a multi-layered elongate tubular body extending from a proximal end to a distal end, the multi-layered, elongate tubular body comprising an outer layer formed from a polymeric material;
a marker element disposed over a portion of the elongate tubular body including the outer layer; and
a first strip of material disposed adjacent to and in contact with a first end of the marker element, wherein the first strip of material is formed from the polymeric material of the outer layer of the multi-layered tubular body defining a mechanical interlock between the marker element and the portion of the elongate tubular body over which it is disposed.

10. The catheter of claim 9, wherein a flexibility of the elongate tubular body increases from a proximal end to a distal end of the tubular body.

11. The catheter of claim 9, wherein the, multi-layered elongate tubular body further comprises an inner layer and a metal braid disposed between the outer layer and the inner layer.

12. The catheter of claim 9, further comprising a second strip of material disposed adjacent to and in contact with a second end of the marker element, wherein the second strip of material is also formed from the polymeric material of the outer layer and contacts the second end of the marker element to further define a mechanical interlock between the marker element to the portion of the elongate tubular body over which it is disposed.

13. The catheter of claim 9, further comprising a distal tip having a proximal end disposed adjacent a second end of the marker element, wherein the proximal end of the distal tip abuts the second end of the marker band defining a bumper formed from a portion of the distal tip so as to mechanically interlock the proximal end of the distal tip with the second end of the marker element.

14. The catheter of claim 13, wherein the proximal end of the distal tip abuts the second end of the marker element and has an outer diameter approximately equal to or less than the marker element.

15. The catheter of claim 9, wherein the marker element is radiopaque.

16. The catheter of claim 9, wherein the marker element is a radiopaque metal marker band that circumscribes the distal portion of the elongate tubular body.

17. A method of securing a marker element to an elongate body, the method comprising:
disposing a marker element on a selected region of the elongate body, the elongate body comprising a polymeric material;
disposing a heat shrink tubing over the selected region of the elongate body including the marker element, wherein the heat shrink tubing extends past at least a first end of the marker body; and
heating the elongate body including the heat shrink tubing disposed over marker element to soften the polymeric material and to constrict the heat shrink tubing in at least a longitudinal direction towards a center line of the marker element, wherein softening of the polymeric material and constriction of the heat shrink tubing forms at least a first strip of material disposed adjacent to and in contact with a first end of the marker element, wherein the first strip of material is formed from the polymeric material of the tubular body defining a mechanical interlock between the marker element and the selected region of the elongate body.

18. The method of claim 17, further comprising positioning a distal tip element comprising a polymeric material adjacent a second end of the marker element, wherein the heat shrink tubing overlaps a proximal end of the distal tip and wherein heating the tubular body including the heat shrink tubing further causes the polymeric material of the distal tip element to soften and abut the second end of the marker element forming a mechanical interlock between the second end of the marker element and the proximal end of the distal tip element.

19. The method of claim 17, wherein the heat shrink tubing further extends past a second end of the marker element, wherein the softening of the polymeric material and the constriction of the heat shrink tubing in response to heating forms a second strip of material disposed adjacent to and in contact with a second end of the marker element, wherein the second strip of material is formed from the polymeric material of the elongate body and defines a mechanical interlock between the marker element and the selected region of the elongate body.

20. The method of claim 17, wherein the step of forming the marker element comprises swaging the marker element onto the selected region of the elongate body.
